(19) United States
(12) Patent Application Publication
(10) Pub. No.: US 2014/0243231 A1
(43) Pub. Date: Aug. 28, 2014

(54) METHODS AND COMPOSITIONS FOR THE EFFICIENT REUSE OF MULTIPLY DYED PARTICLES

(71) Applicant: The Terasaki Family Foundation, Los Angeles, CA (US)

(72) Inventors: Junchao Cai, Torrance, CA (US); Wei Huang, Los Angeles, CA (US)

(21) Appl. No.: 14/192,804

(22) Filed: Feb. 27, 2014

Related U.S. Application Data
(60) Provisional application No. 61/770,996, filed on Feb. 28, 2013.

(51) Int. Cl. G01N 33/543; C12Q 1/68; G01N 33/58

(52) U.S. Cl. CPC G01N 33/54393; G01N 33/583; C12Q 1/6834
USPC 506/9; 436/501; 435/6.11

(57) ABSTRACT

Provided herein are compositions and methods for reuse and/or recycling of internally dyed particles useful for multiplex assays of target analytes.

### CROSS-REFERENCE TO RELATED APPLICATION

This application claims the benefit of priority of U.S. Provisional Application No. 61/770,996, filed Feb. 28, 2013, the contents of which are hereby incorporated by reference in their entirety.

### FIELD

Provided herein are methods and compositions for the efficient reuse of multiply dyed particles. In useful embodiments, the costly multiply dyed particles can be used two, three or more times to provide more efficient and economic use of the particles.

### BACKGROUND

Multiple analytes or targets can be distinguished in a single sample simultaneously using commercially available particles that are labeled with one, two or more labels. Particularly useful particles comprise internal dyes, for instance two or more fluorescent internal dyes, in each particle. The multiple internal dyes can be used to uniquely label particular particles in a mixture. Binding to a particle can be identified by detecting the unique label and a label for binding, for instance, a reporter dye. Using this system of multiplex particle labels and reporter labels, complex samples can be assayed with up to 500 or more unique bioassays simultaneously in the same reaction.

Building an assay on the multiply dyed particles is a simple, easy process. Multiply dyed particles are commercially available, as are capture molecules useful for capturing target analytes. Useful capture molecules include peptides, proteins and polynucleotides. Many of the multiply dyed particles are prepared for covalent linking to capture molecules. Certain multiply dyed particles already bound to capture molecules are also commercially available.

Currently, commercial supplier Luminex offers two lines of beads (uncoupled and coupled) for development of protein or oligonucleotide related assays on the Luminex® platform. Uncoupled microsphere products, including MicroPlex®, MagPlex®, and SeroMAP™ microspheres, have carboxylated surfaces and are ready for 1-ethyl-3-(3-dimethylaminopropyl)carbodiimide (EDC)-mediated coupling of proteins or amine modified oligonucleotides. They can be used for any custom protein, peptide or polynucleotide assay. Coupled microspheres, including MagPlex®-TAG and LumAvidin® Microspheres, are coupled to proteins, peptides or oligonucleotides for specific nucleic acid applications or peptide and protein assays. Luminex's bead-based assay platform has been widely used in detecting proteins, peptides and polynucleotides.

Unfortunately, commercially available multiply dyed particles are expensive, and to date have been generally limited to single use applications. One buys the multiply dyed particles at a high price, uses the particles in a single assay, and then discards the used particles. This has resulted in high use of material and financial resources by those of skill in the art. There is a strong need for methods and compositions that provide reliable, efficient, repeatable cleaning and reuse of used multiply dyed particles.

### SUMMARY

Provided herein are compositions and methods useful for cleaning and reusing internally dyed particles. In certain embodiments, the compositions are effective to separate an internally dyed particle with capture molecules from target analytes. In certain embodiments, the methods are effective for providing internally dyed particles with capture molecules separated from target analytes and ready for use with additional target analytes. In use, the compositions provided herein provide for the use of internally dyed particles with capture molecules two or more times, three or more times, four or more times or more.

In one aspect, provided herein are methods of recycling an internally dyed particle with capture molecules that are bound to one or more analytes. The method comprises contacting the internally dyed particles with capture molecules with a composition effective to disrupt the bonds between the capture molecules on the particle and the one or more analytes. The composition can be any composition capable of disrupting the bonds. In certain embodiments, the composition comprises a disrupting element selected from the group consisting of acid, base, ionic strength and heat sufficient to disrupt the bonds between the capture molecules on internally dyed particle and the one or more analytes.

In another aspect, provided herein are methods of cleaning an internally dyed particle with capture molecules that are bound to one or more analytes. The method comprises contacting the internally dyed particles with a composition effective to disrupt the bonds between the capture molecules on the particle and the one or more analytes. The composition can be any composition capable of disrupting the bonds. In certain embodiments, the composition comprises a disrupting element selected from the group consisting of acid, base, ionic strength and heat sufficient to disrupt the bonds between the capture molecules on internally dyed particle and the one or more analytes.

In another aspect, provided herein are methods of assaying analytes with internally dyed particles. The methods of assaying comprise the steps of assaying a first analyte or mixture of analytes with the particles, cleaning the particles according to any technique described herein, and assaying a second analyte or mixture of analytes with the cleaned particles.

In another aspect, provided herein are compositions useful for cleaning or reusing multiply dyed particles. The compositions comprise an internally dyed particle with capture molecules, one or more analytes capable of binding the capture molecules on the internally dyed particle and a composition effective to disrupt the bonds between the capture molecules on the particle and the one or more analytes. The composition can be any composition capable of disrupting the bonds. In certain embodiments, the composition comprises a disrupting element selected from the group consisting of acid, base, ionic strength and heat sufficient to disrupt the bonds between the capture molecules on internally dyed particle and the one or more analytes.

In another aspect, provided herein are methods of recycling internally dyed, analyte-bound particles comprising the step of: contacting the internally dyed, analyte-bound particles with a sufficient amount of acid, a sufficient amount of base, a sufficient ionic strength or sufficient heat, or a combination thereof, to separate the internally dyed particles from the analyte to yield recycled, internally dyed particles substantially free of analyte.

In some embodiments, the recycled, internally dyed particles are free of analyte.

In some embodiments, the sufficient amount of acid yields a pH of about 2.3.

In some embodiments, the contacting step is performed for about 1 to about 30 minutes.

In some embodiments, the recycled, internally dyed particles bind at least about 75% of the analyte bound by the internally dyed particles under the same conditions.

In some embodiments, the first analyte and the second analyte are the same analyte. In another aspect, the first analyte and second analyte are different analytes.

In another aspect, provided herein are methods of recycling internally dyed, analyte-bound particles comprising separating the internally dyed particles from the analyte to yield recycled internally dyed particles substantially free of analyte, wherein the recycled internally dyed particles bind at least about 75% of the analyte bound by the internally dyed particles under the same conditions.

In some embodiments, the recycled internally dyed particles bind at least about 85% of the analyte bound by the internally dyed particles under the same conditions.

In another aspect, provided herein are kits for recycling internally dyed, analyte-bound particles comprising: a composition for separating internally dyed particles from an analyte, said composition comprising a substance selected from the group consisting of an acid, a low pH buffer, a base, a high pH buffer, an electrolyte, a chaotrope, and a combination thereof; instructions for recycling the internally dyed, analyte-bound particles using at least one composition recited above.

In some embodiments, the kit comprises a container for treating the internally dyed, analyte-bound particles.

In some embodiments, the kit comprises a device for separating the treated particles from the analyte.

In some embodiments, the device comprises a magnet or a magnetized container.

The compositions and methods herein provide the cleaning and reuse of internally dyed particles with capture molecules. According to these compositions and methods, the internally dyed particles with capture molecules can be used more than one time, in certain embodiments, more than two times, more than three times, more than four times or even more times. The compositions and methods are based, in part, on the discovery that the internally dyed particles with capture molecules that have conventionally been discarded after a single use can, in fact, be cleaned and reused effectively.

### BRIEF DESCRIPTION OF THE FIGURES

FIG. 1 provides an exemplary particle capturing an exemplary target analyte;

FIG. 2 provides an cartoon of an example demonstrating the reuse of antigen capture particles;

FIG. 3 provides dissociation of antigen from particles;

FIG. 4 provides binding with original and reused antigen capture particles;

FIG. 5 provides a cartoon of reuse of α-HLA antibody capture particles;

FIG. 6 provides dissociation of antibody from particles;

FIG. 7. provides binding with original and reused α-HLA antibody capture particles

FIG. 8 provides a cartoon of an oligonucleotide capture particle;

FIG. 9 provides dissociation of target oligonucleotide from a capture particle; and

FIG. 10 provides binding with original and reused oligonucleotide capture particles.

### DESCRIPTION OF EXEMPLARY EMBODIMENTS

Provided herein are compositions and methods for reuse and recycling of internally dyed particles for the capture of target analytes.

### Definitions

When referring to the compositions and methods provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

The term “about” indicates and encompasses an indicated value and in addition a range above and below that value. In certain embodiments, the term about indicates the designated value ±10%, ±5% or ±1%. In certain embodiments, the term about indicates the designated value ±one standard deviation of that value. When applied to logarithmic values such as pH values, the term indicates ±0.2 or ±0.1 on the appropriate logarithmic scale.

Recycled, internally dyed particles may be substantially free of analyte. The term “substantially free of,” when used with respect to recycled, internally dyed particles and an analyte, means that the analyte that was bound to the internally dyed particles before treatment (e.g., with acid, base, ionic strength, heat, or other treatment) is substantially absent from the particles after treatment (i.e., the recycled particles). In some embodiments, the amount of analyte bound to the particles after treatment may be about 1%, about 5%, about 10%, about 15%, about 20%, or about 25% of the analyte that was bound to the particles before treatment. In some embodiments, the amount of analyte bound to the particles after treatment may be less than 1%, less than 5%, less than 10%, less than 15%, less than 20%, or less than 25% of the analyte that was bound to the particles before treatment. The amount of analyte bound to the particles before and after treatment may be measured by any suitable assay including, but not limited to, a fluorescence assay. One of ordinary skill in the art will recognize that the amount of analyte bound to the particles after treatment may vary according to the affinity and avidity between the capture molecule on the particle and the analyte.

The term “isolated” with respect to a composition refers to a composition that includes at least 85%, 90%, 95%, 98%, 99% to 100% by weight, of a substance, the remainder comprising other substances.

Methods

Provided herein are methods for cleaning or recycling internally dyed particles with capture molecules. The methods can be used to provide particles that can be used two or more times for binding and/or detecting target analytes. One of ordinary skill in the art will recognize that the methods provided herein are applicable to beads binding a single analyte or beads binding multiple analytes.

In the methods, capture molecules on an internally dyed particle that are bound to analytes are contacted with an element selected from the group consisting of acid, base, high ionic strength and heat, and combinations thereof, in a sufficient amount and for a sufficient time to disrupt the bond between the capture molecules on the particle and the analytes. Following disruption, the internally dyed particle with capture molecules can be isolated from the analytes according to any technique apparent to a practitioner of skill. The resulting cleaned or recycled internally dyed particle with capture molecules can be used for any purpose deemed useful by the practitioner of skill.

As shown in the examples below, the cleaned or recycled internally dyed particles with capture molecules show binding comparable to unused internally dyed particles. The binding is specific, measureable and repeatable. According to the methods provided herein, the costly internally dyed particles can be used two or more times to provide more efficient use of financial and material resources.

In certain embodiments, the cleaning or recycling can be achieved with an acid condition. In the methods, the acid condition can be any acid condition deemed useful by the practitioner of skill to disrupt the bond between the target analyte and the capture molecules on internally dyed particle. The acid condition should be sufficient to disrupt binding without damaging the internally dyed particle and any molecules covalently bound to its surface. In certain embodiments, the acid condition is pH 2.0-3.6. In certain embodiments, the acid condition is pH 2.0-3.0. In certain embodiments, the acid condition is pH 2.2-3.6. In certain embodiments, the acid condition is pH 2.5-3.0. In certain embodiments, the acid condition is pH 3.0-6.2. In certain embodiments, the acid condition is pH 3.6-5.6. In certain embodiments, the acid condition is pH 5.0-6.6. In certain embodiments, the acid condition is about pH 2.3. In certain embodiments, the acid condition is about pH 2.8. In certain embodiments, the acid condition is about pH 3.0. In certain embodiments, the acid condition is about pH 6.6. The acid condition at the desired pH can be reached or maintained by any technique apparent to those of skill in the art. In certain embodiments, the acid condition can be reached or maintained with an appropriate pH buffer deemed useful by those of skill in the art. Useful pH buffers include glycine, citrate, acetate, cacodylate, maleate, phosphate, glycylglycine, malate, formate, and succinate, combinations thereof, or any salt thereof. Useful salts include HCl salts, for instance glycine-HCl. The buffer can be used at any concentration deemed suitable by the practitioner of skill. In certain embodiments, the pH buffer is at a concentration of 25-200 mM, 25-150 mM or 50-150 mM. Useful buffer concentrations include about 25 mM, about 50 mM, about 75 mM, about 100 mM, about 125 mM and about 150 mM. The acid condition can also be reached with an acid such as HCl or H₂SO₄, with or without the use of a buffer. The acid condition can be used by itself, or in combination with the ionic strength and/or heat condition.

In certain embodiments, the cleaning or recycling can be achieved with a base condition. In the methods, the base condition can be any base condition deemed useful by the practitioner of skill to disrupt the bond between the target analyte and the capture molecule on internally dyed particle. The base condition should be sufficient to disrupt binding without damaging the internally dyed particle and any molecules covalently bound to its surface. In certain embodiments, the base condition is pH 8.6-10.6. In certain embodiments, the base condition is pH 8.6-10.0. In certain embodiments, the base condition is pH 8.6-9.5. In certain embodiments, the base condition is about pH 10-11.5. In certain embodiments, the base condition is about pH 11-11.7. In certain embodiments, the base condition is pH 11.7-14. In certain embodiments, the base condition is about pH 10. In certain embodiments, the base condition is about pH 10.5. In certain embodiments, the base condition is about pH 11. In certain embodiments, the base condition is about pH 11.5. In certain embodiments, the base condition is about pH 12. In certain embodiments, the base condition is about pH 12.7. In certain embodiments, the base condition is about pH 13. In certain embodiments, the base condition is about pH 13.3. In certain embodiments, the base condition is about pH13.7. In certain embodiments, the base condition is about pH 14. The base condition at the desired pH can be reached or maintained by any technique apparent to those of skill in the art. In certain embodiments, the base condition can be reached or maintained with an appropriate pH buffer deemed useful by those of skill in the art. Useful pH buffers include glycine, sodium borate, diethylamine, triethylamine, triethanolamine, ammonium hydroxide, tricine, glycylglycine, Tris, HEPPS, EPPS (N-(2-hydroxyethyl)-piperazine-N′-3-propanesulfonic acid), bicine, TAPS (3-{[tris(hydroxymethyl)-methyl]-amino}-propanesulfonic acid), AMPD (2-amino-2-methyl-1,3-propanediol, ammediol), AMPSO (N-(1,1-dimethyl-2-hydroxyethyl)-3-amino-2-hydroxypropanesulfonic acid), taurine, ammonia, CHES (cyclohexylaminoethanesulfonic acid), AMP (2-amino-2-methyl-1-propanol), CAPSO (3-(cyclohexylamino)-2-hydroxy-1-propanesulfonic acid), carbonate and CAPS (3-(cyclohexylamino)-propanesulfonic acid), combinations thereof, or any salt thereof. Useful salts include NaOH salts, for instance glycine-NaOH. The buffer can be used at any concentration deemed suitable by the practitioner of skill. In certain embodiments, the pH buffer is at a concentration of 25-200 mM, 25-150 mM or 50-150 mM. Useful buffer concentrations include about 25 mM, about 50 mM, about 75 mM, about 100 mM, about 125 mM and about 150 mM. The base condition can also be reached with a base such as NaOH, with or without the use of a buffer. Useful base conditions include 0.005-1.0 M NaOH, 0.005-0.75 M NaOH and 0.005-0.5 M NaOH. The base condition can be used by itself, or in combination with the ionic strength and/or heat condition.

In certain embodiments, the cleaning or recycling can be achieved with a high ionic strength condition. In the methods, the ionic strength condition can be any ionic strength condition deemed useful by the practitioner of skill to disrupt the bond between the target analyte and the capture molecule on internally dyed particle. The ionic strength condition should be sufficient to disrupt binding without damaging the internally dyed particle and any molecules covalently bound to its surface. In certain embodiments, the ionic strength condition is 0.2-5 M, 0.5-5 M, 0.5-4 M, or 0.5-3 M. The ionic strength condition can be reached or maintained by any technique apparent to those of skill in the art. In certain embodiments, the base condition can be reached or maintained with an appropriate salt deemed useful by those of skill in the art. Useful salts include lithium chloride, magnesium chloride, potassium chloride, sodium chloride, sodium iodide, potassium iodide, sodium thiocyanate, and combinations thereof. Particularly useful ionic strength conditions include 5 M lithium chloride, 3.5 M magnesium chloride, 3.5 M potassium chloride, 3.0 M potassium chloride, 2.5 M sodium chloride, 2.5 M potassium iodide, 2 M sodium chloride, and 0.2-3.0 M sodium thiocyanate. The ionic strength condition can be used by itself or in combination with the acid condition, base condition or heat condition.

In certain embodiments, the cleaning or recycling can be achieved with a heat condition. In the methods, the heat condition can result in any temperature deemed useful by the practitioner of skill to disrupt the bond between the target analyte and the capture molecule on internally dyed particle. Heat can be particularly useful for internally dyed beads coupled to polynucleotide capture molecules and polynucleotide target molecules. The heat condition should result in a temperature above the melting temperature (Tm) of the capture molecule-target molecule complex and lower than the highest temperature tolerated by the beads. In certain embodiments, the internally dyed beads and target analyte are heated to a temperature of 60-80° C., 60-85° C., 70-90° C., 70-95° C., 75-95° C., or 85-100° C. The heat condition can be reached or maintained by any technique apparent to those of skill in the art. The heat condition can be used by itself or in combination with the acid condition, base condition or ionic strength condition.

The above conditions can be used individually, in combination or in sequence. The practitioner of skill should be able to identify which condition is appropriate to each internally dyed bead—target analyte. In most cases, the acid condition should be sufficient. In other cases, the base condition should be sufficient. If neither the acid nor base condition is sufficient, the high ionic strength condition would be used. Heat is particularly useful for polynucleotide capture molecule—target analyte complexes.

Some capture molecules, such as proteins or protein complexes with multiple subunits, may be denatured by certain treatment conditions used to clean the beads. If this denaturation occurs, and if binding of an analyte to the capture molecule is dependent upon the conformation of the capture molecule, then renaturation of the capture molecule may be required before the treated beads can be re-used.

Any or all of the above conditions can further include a chaotropic agent such as urea to facilitate separation of the particles from the target analyte. Useful chaotropic agents include ethanol, butanol, lithium acetate, lithium perchlorate, lithium acetate, magnesium chloride, phenol, propanol, sodium dodecyl sulphate, dimethyl sulfoxide, formamide, glyoxal, urea, thiourea, guanidine, and combinations thereof. (e.g. 2-6M guanidine-HCl, 2-8M urea). Some of these agents are particularly useful when heat is the dissociative condition.

Any or all of the above conditions can be applied for a defined time. The optimum time will generally be the shortest time sufficient to yield recycled, internally dyed particles substantially free of analyte. In some embodiments, the time may be about 1 minute, about 5 minutes, or about 30 minutes. In some embodiments, the time may be about 1 to about 30 minutes; about 1 to about 5 minutes, about 5 to about 10 minutes, about 10 to about 20 minutes, or about 20 to about 30 minutes. In some embodiments, the time may be less than 1 minute, less than 5 minutes, less than 10 minutes, less than 20 minutes, or less than 30 minutes.

In some embodiments, the ability of particles to bind an analyte after treatment may be compared to the ability of particles to bind the same analyte before treatment. This comparison should be performed under the same conditions, for example by maintaining the same medium composition, analyte concentration, and particle concentration. In some embodiments, the treated particles bind at least about 75% of the analyte bound by the pre-treatment particles under the same conditions. In some embodiments, the treated particles bind at least about 85% of the analyte bound by the pre-treatment particles under the same conditions. In some embodiments, the treated particles bind at least about 90% of the analyte bound by the pre-treatment particles under the same conditions. In some embodiments, the treated particles bind at least about 95% of the analyte bound by the pre-treatment particles under the same conditions. In some embodiments, the treated particles bind at least about 99% of the analyte bound by the pre-treatment particles under the same conditions. In some embodiments, the treated particles bind at least about 100% of the analyte bound by the pre-treatment particles under the same conditions (i.e., the treated particles bind more analyte than the pre-treatment particles).

The internally dyed particles are particles well-known to those of skill in the art. The particles typically comprise a matrix material and an internal dye. Preferred particles comprise a matrix material and two or more internal dyes useful for labeling the particles in a multiplex mixture. The matrix material is typically polystyrene. The matrix material can be in any form, for example, in the form of microspheres. Useful dyes include fluorescent dyes. The matrix material is generally bound to a capture molecule that is capable of specifically binding a target analyte. In certain embodiments, the particles are magnetic. Useful capture molecules include proteins, antibodies, peptides and polynucleotides. The particles are commercially available with activated surfaces capable of forming covalent bonds to one or more capture molecules. The particles are also commercially available with surfaces that are already covalently bonded to one or more capture molecules. Useful internally dyed particles include those described in U.S. Pat. Nos. 6,514,295, 6,268,222, 6,528,165, 6,592,822, 6,599,331, 6,632,526, 6,649,414, 6,916,661, 6,929,859, 7,141,431, 7,445,844, 7,718,262, 8,038,734, 8,088,629, 8,283,037 and 8,361,169, the contents of each of which are hereby incorporated by reference in their entireties. In preferred embodiments, the internally dyed particles are selected from the group consisting of MicroPlex®, MagPlex® and SeroMAP™ microspheres, each available from Luminex Corp. (Austin, Tex.). In preferred embodiments, the internally dyed particles are selected from the group consisting of MagPlex-TAG® and LumAvidin® microspheres, each available from Luminex Corp. (Austin, Tex.).

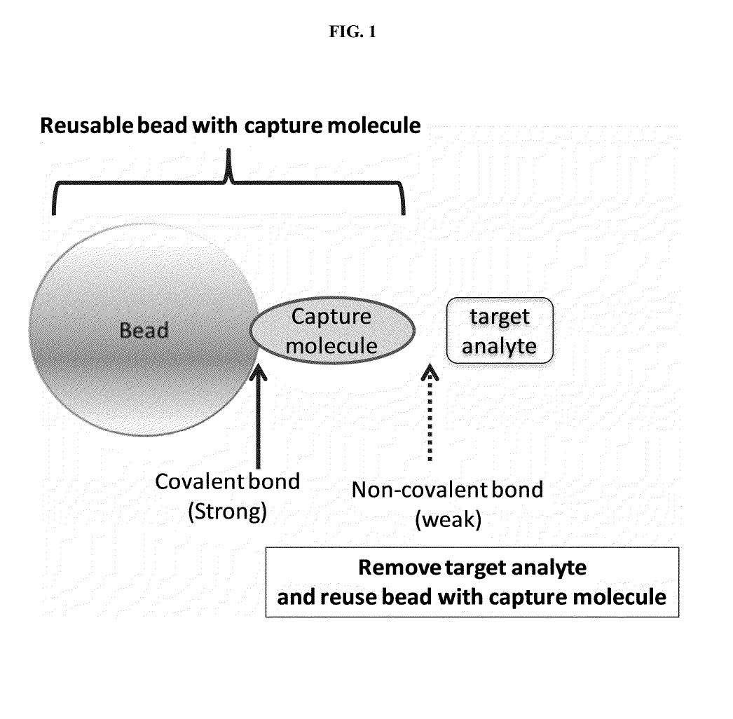

The internally dyed particles with a covalent capture molecule can be used to capture any target analyte deemed suitable to practitioners of skill without limitation. As shown in FIG. 1, an internally dyed Bead is covalently linked to a Capture Molecule. The Capture Molecule can be used to bind a Target Analyte in a first assay. The Capture Molecule—Target Analyte binding pair can be any binding pair without limitation. Useful capture molecules include proteins, antibodies, peptides and polynucleotides. Useful Target Analytes also include proteins, antibodies, peptides and polynucleotides that bind the respective Capture Molecule.

Compositions

Also provided herein are compositions useful for cleaning, reusing and recycling internally dyed particles with capture molecules for the capture of target analytes.

The compositions comprise an internally dyed particle as described above along with a target analyte for the particle. The compositions further comprise a disruption element sufficient to disrupt the bond between the capture molecule on the particle and the target analyte without disrupting the structure or function of the internally dyed particle and any capture molecule bound to the particle. In certain embodiments, the composition comprises a condition selected from the group consisting of an acid condition, a base condition, an ionic strength condition, a heat condition, and combinations thereof. The conditions are described in detail above. The compositions can further comprise any element deemed useful for the practitioner of skill without limitation. Examples include preservatives, buffers, water, phosphate, etc.

The compositions can be prepared by any technique deemed suitable by those of skill in the art. The particles can be obtained from commercial sources. The covalently bound capture molecule can be prepared by standard techniques or obtained commercially. In certain embodiments, the particles can be obtained commercially with capture molecules covalently bound. The target analyte can be prepared or obtained by any technique without limitation. The acid conditions, base conditions, ionic strength conditions and heat conditions are well within the ordinary skill and resources of practitioners in the art.

Kits

Also provided are kits for useful for cleaning, reusing or recycling internally dyed particles. The kits comprise compositions useful for providing the disrupting conditions described above. For instance, the kits can comprise an acid, a low pH buffer, a base, a high pH buffer, and/or an ionic strength composition. The kits can further comprise a container for cleaning the particles. The kits can also further comprise a device useful for separating the cleaned particles from the target analytes, for instance a magnet or magnetized container for magnetic internally dyed particles. In some embodiments, suitable packaging is provided. As used herein, “packaging” includes a solid matrix or material customarily used in a system and capable of holding within fixed limits a composition provided herein. Such materials include glass and plastic (e.g., polyethylene, polypropylene, and polycarbonate) bottles, vials, paper, plastic, and plastic-foil laminated envelopes and the like. The kits may further comprise instructions for cleaning, reusing or recycling internally dyed particles. The instructions may direct one to clean, reuse or recycle the internally dyed particles according to the methods provided in this disclosure.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); and μM (micromolar). Unless otherwise indicated, all temperatures are expressed in ° C. (degrees Centigrade). All reactions are conducted at room temperature unless otherwise noted.

### Example 1

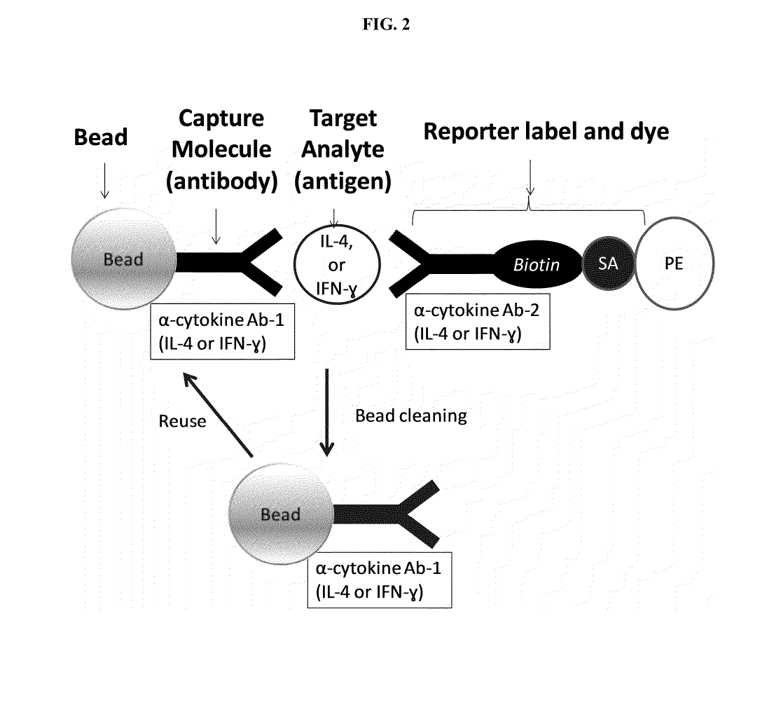

This Example demonstrates the cleaning and reuse of antibody-coupled beads for capture of cytokine antigens. The particles used were Luminex® beads coupled to anti-cytokine (IL-4 or IFN-γ) monoclonal antibodies (MILLIPORE, MILLIPLEX MAP Human Cytokine/Chemokine Panel (MPXHCYTO-60K-02). FIG. 2 provides a scheme of the protocol.

Beads were first contacted with cytokine, a second biotinylated antibody and streptavidin PE (SAPE). Beads were treated using low pH buffer (0.1 M glycine-HCl, pH2.3) for 1, 5, and 30 min respectively (protocol #1, 2, and 3). Phosphate buffered saline (PBS) was used as negative control, while a known concentration of cytokine (1,000 pg/ml) was used as positive control.

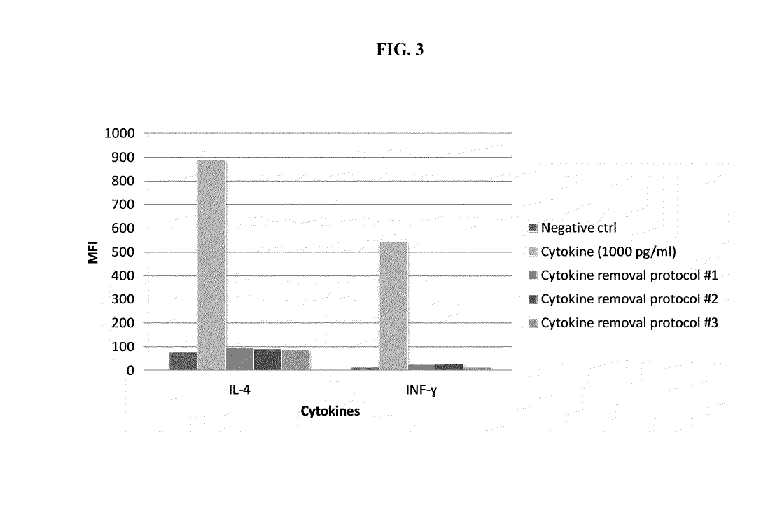

As shown in FIG. 3, after treatment, cytokine, the second antibody, and streptavidin PE (SAPE) were dissociated from the beads. Used beads with positive reaction were collected and treated to remove bound cytokines. For each of protocols #1, 2 and 3, cytokine signals were reduced close to negative control level.

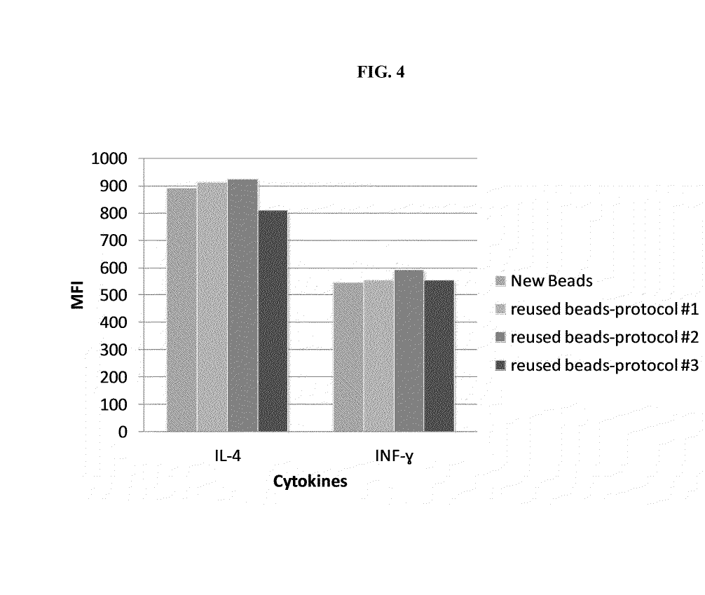

As shown in FIG. 4, treated beads work equally well as new beads in cytokine detection. Cleaned beads using low pH buffer were reused to detect cytokine with known concentration. The mean fluorescence intensities of used beads were similar to that of new beads in detecting the same concentration of target cytokines.

### Example 2

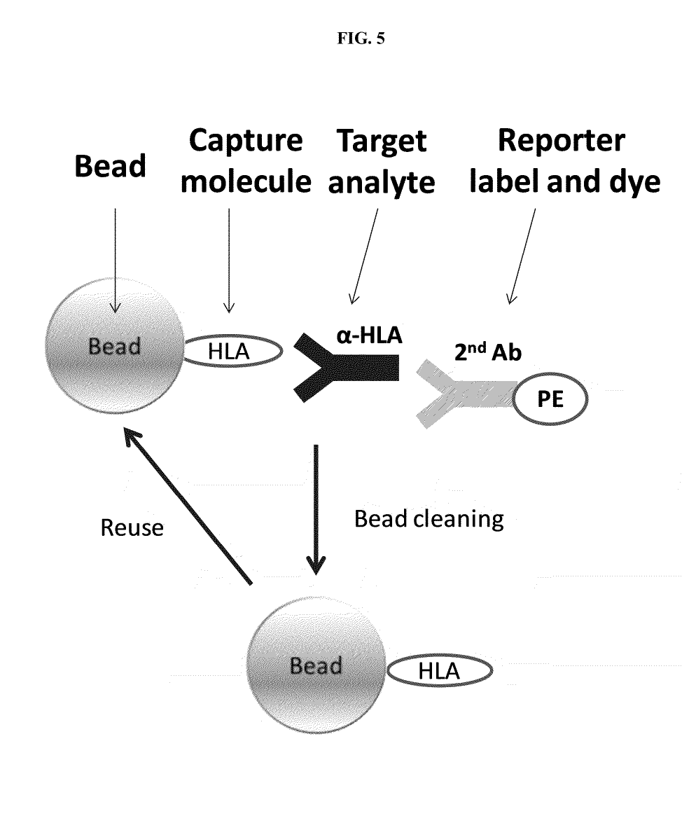

This example demonstrates the successful cleaning of HLA-antigen beads with low pH buffer and successful reuse in binding assays. In this example, HLA class I antigen-coupled beads were cleaned and reused for α-HLA antibody detection. The beads were Luminex® beads coupled to HLA class I antigen (One Lambda Inc., LABScreen® HLA single antigen beads, LS1A04). FIG. 5 provides a scheme for the cleaning and reuse protocol.

HLA-coupled Luminex® beads are commercially available (One Lambda, Inc.) for detection of HLA specific antibodies. If a sample contains HLA antibodies (α-HLA), α-HLA will bind to the HLA antigen on the beads, and a positive signal will be detected by adding PE conjugated 2nd anti-human Ig.

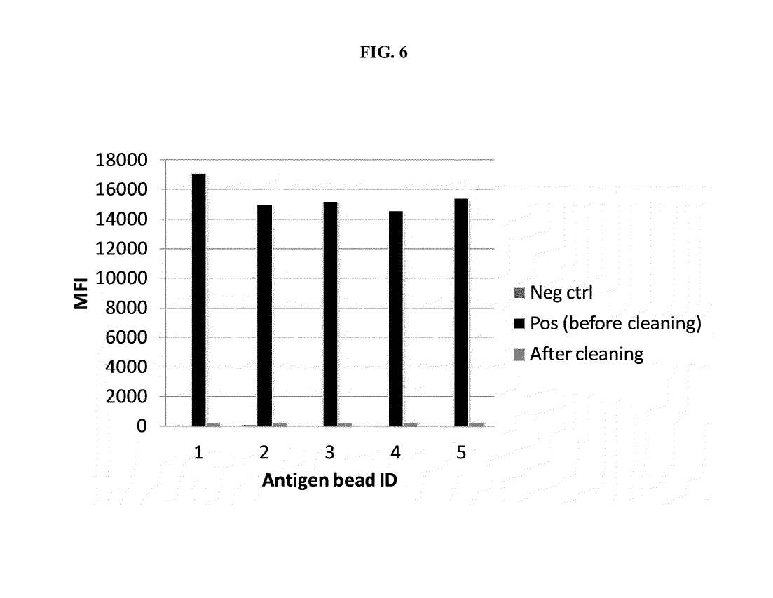

Used beads with positive signal were cleaned using low pH buffer (0.1M glycine-HCl, pH2.3) to remove captured α-HLA. As shown in FIG. 6, after treatment, α-HLA antibody, the secondary PE-conjugated anti-human Ig were dissociated from the beads. Anti-HLA antibody signals were reduced close to negative control level. The beads were then reused to bind and detect α-HLA antibody. This process was repeated five times.

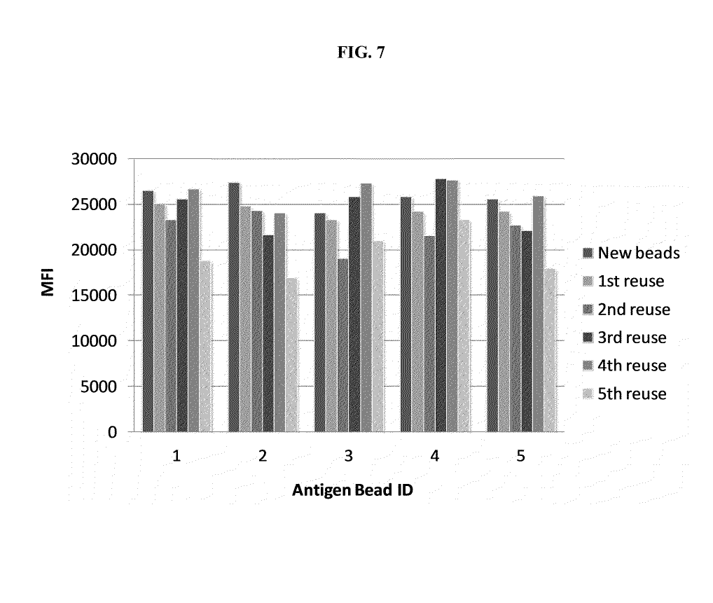

As shown in FIG. 7, HLA antigen beads can be repeatedly used in detection of α-HLA antibodies. A pan-reactive α-HLA monoclonal antibody (HC-10) specific for β2m-free heavy chain of HLA class I antigen was used as a positive control. Used beads worked equally well in detecting the same monoclonal antibody target even after multiple reuses.

### Example 3

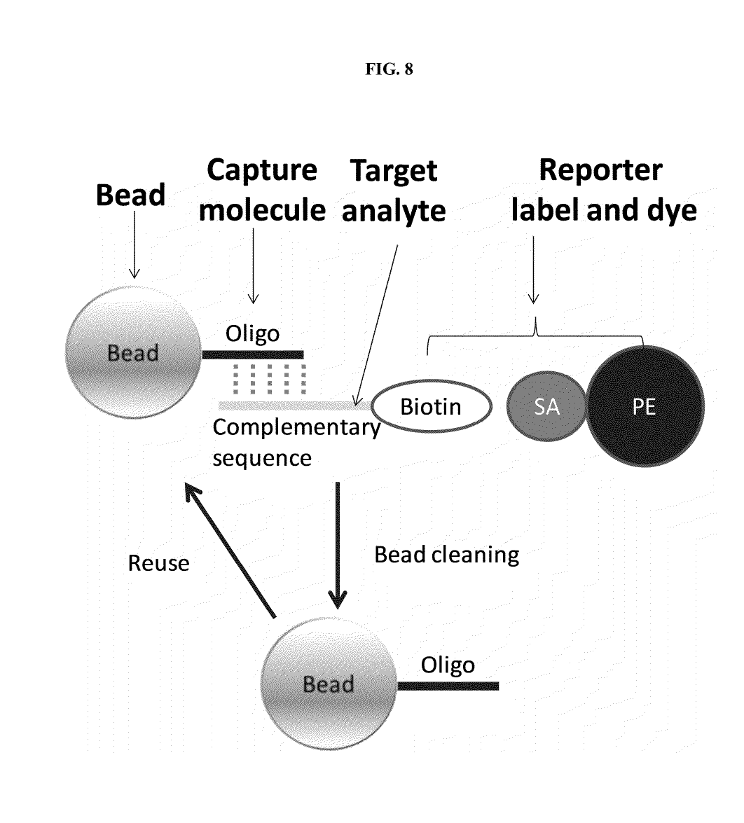

This example demonstrates the use and reuse of beads for the capture of oligonucleotides. As shown in FIG. 8, in order to detect specific nucleic acid sequence, a sequence specific oligonucleotide (SSO) probe with complementary sequence of a target oligonucleotide was covalently coupled to the bead (One Lambda Inc., LABType® SSO for HLA typing). The interaction between SSO probe and its complementary target was non-covalent, which included hydrogen bonds between nucleotides and base-stacking interactions among aromatic nucleobases.

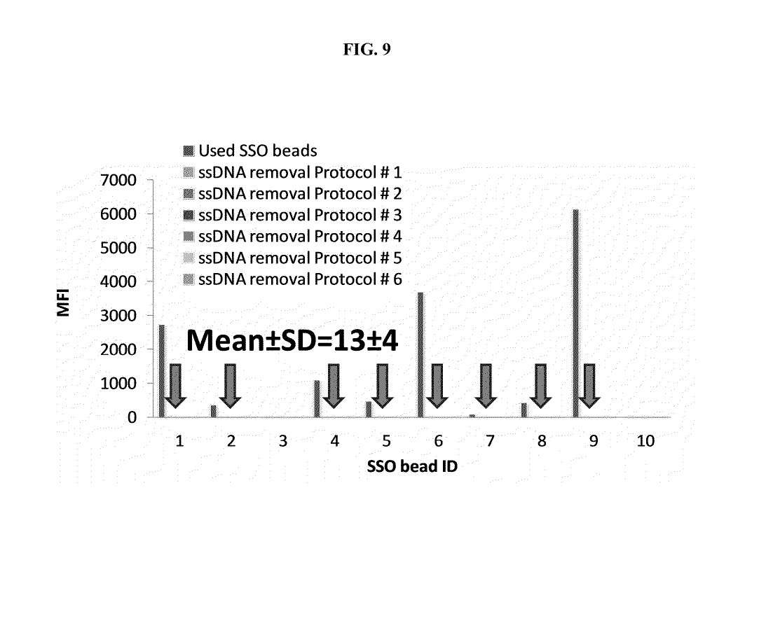

As shown in FIG. 9, captured target single-stranded DNA was successfully dissociated from oligonucleotide-coupled beads. Eight out of 10 used (i.e., recycled) SSO beads (#1, 2, 4, 5, 6, 7, 8, and 9) had detectable complimentary oligonucleotides (ssDNA) bound to the beads. The mean fluorescence intensities (MFI) of these reactions varied between 90 to 6000. After treating the used beads with heating (Protocol #1-3, 1-8M urea added) and NaOH (Protocol #4-6, 0.2-0.5M NaOH), all captured target ssDNA were successfully dissociated from the bead surfaces. The average MFI level of treated beads was 13±4.

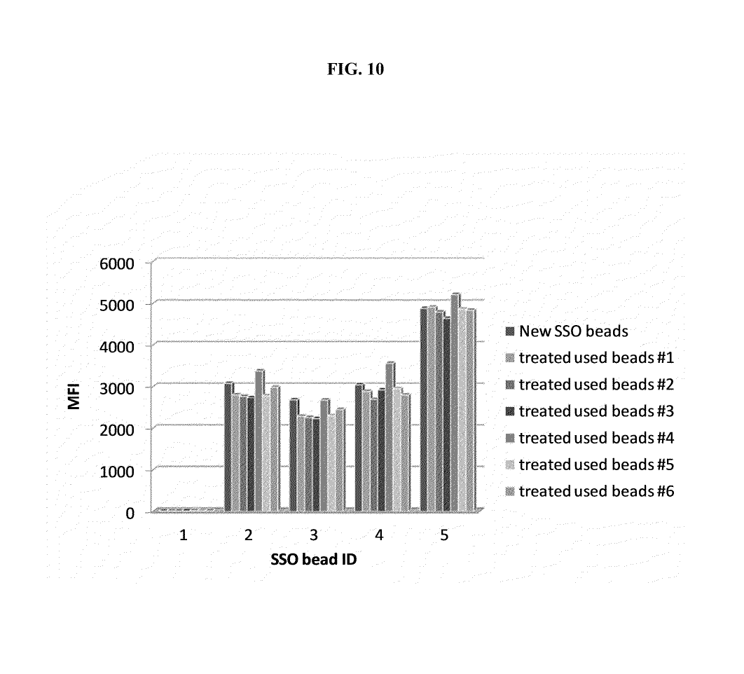

As shown in FIG. 10, used SSO beads worked equally well as new beads in SSO typing. Used SSO typing beads were collected and cleaned using 6 different protocols. Treated beads were then used for new sample testing. Compared with new SSO beads in testing the same DNA sample, used beads generated with the 6 treatment protocols showed the same reaction patterns and similar MFI levels.

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

What is claimed is:

1. A method of recycling internally dyed, analyte-bound particles comprising the step of:
contacting the internally dyed, analyte-bound particles with a sufficient amount of acid, base, ionic strength or heat, or a combination thereof, to separate the internally dyed particles from the analyte to yield recycled, internally dyed particles free of analyte.

2. The method of claim 1 wherein said internally dyed, analyte-bound particles are microspheres.

3. The method of claim 1 wherein said internally dyed, analyte-bound particles are microspheres, each comprising two or more fluorescent dyes.

4. The method of claim 1 wherein said internally dyed, analyte-bound particles are Luminex™ beads.

5. The method of claim 1 wherein said analyte is selected from the group consisting of proteins, peptides and polynucleotides.

6. The method of claim 1 wherein said sufficient amount of acid yields a pH of 2.0-6.6.

7. The method of claim 1 wherein said sufficient amount of base yields a pH of 8.6-14.

8. The method of claim 1 wherein said ionic strength is 200 mM to 5 M.

9. The method of claim 1 wherein said sufficient amount of heat yields a temperature between the melting temperature of the analyte-particle complex and 100° C.

10. A method of assaying analyte binding comprising the steps of:
a. contacting internally dyed particles with a first analyte;
b. measuring first analyte binding;
c. recycling the internally dyed, analyte-bound particles according to claim 1;
d. contacting the recycled internally dyed particles with a second analyte; and
e. measuring second analyte binding.

11. A composition comprising internally dyed particles, analyte and a sufficient amount of acid, base, electrolyte or heat to separate the internally dyed particles from the analyte.
